(19) United States
(12) Patent Application Publication
(10) Pub. No.: US 2026/0368851 A1
(43) Pub. Date: Nov. 19, 2026

(54) NON-LINEAR COLOR DEMULTIPLEXING BY AUTOENCODER FRAMEWORK

(71) Applicant: VENTANA MEDICAL SYSTEMS, INC., Tucson, AZ (US)

(72) Inventors: Jim F. Martin, Mountain View, CA (US); Nahil Atef Sobh, Urbana, IL (US)

(73) Assignee: VENTANA MEDICAL SYSTEMS, INC., Tucson, AZ (US)

(21) Appl. No.: 19/726,635

(22) Filed: Jun. 30, 2026

Related U.S. Application Data
(63) Continuation of application No. PCT/US2024/062290, filed on Dec. 30, 2024.
(60) Provisional application No. 63/621,690, filed on Jan. 17, 2024.

(51) Int. Cl. G06T 3/4015; G06N 3/0455; G06V 10/774; G16B 40/10

(52) U.S. Cl. CPC G06T 3/4015; G06N 3/0455; G06V 10/774; G16B 40/10

(57) ABSTRACT

The present disclosure relates to robust color demultiplexing of multiplex immunohistochemistry (mIHC) images using an autoencoder-based machine-learning (ML) framework trained on a diverse dataset that may include one or more chromogenic dyes comprising different multiplex digital pathology images. The machine-learning model may include an encoder (demultiplexing) network that was trained with a decoder (multiplexing) network configured to transform the set of synthetic singleplex images into a synthetic multiplex image. The model is trained using a loss function configured such that a calculated loss minimizes a difference between input multiplex digital pathology images and synthetic multiplex images. The trained machine-learning model generates a set of synthetic singleplex images, where each synthetic singleplex image corresponds to an IHC marker. The encoder (demultiplexing) network may be configured to generate the set of synthetic singleplex images conditioned on a number of IHC markers from the multiplex digital pathology image using an attention mechanism. The encoder network may be trained using a loss function configured such that a calculated loss scales with a degree of difference between the set of synthetic singleplex images and a set of reference singleplex images, wherein the set of reference singleplex images are adjacent tissue slides stained using single IHC marker of the one or more IHC markers.

### CROSS-REFERENCE OF RELATED APPLICATIONS

This application is a continuation of International Patent Application No. PCT/US2024/062290, filed on Dec. 30, 2024, which claims priority to U.S. Provisional Patent Application No. 63/621,690, filed on Jan. 17, 2024, titled “NON-LINEAR COLOR DEMULTIPLEXING BY AUTOENCODER FRAMEWORK,” which is incorporated by reference in its entirety for all purposes.

### BACKGROUND

In histopathology, diseased tissue is analyzed by microscopic examination of stained tissue sections. Histological staining is a technique that enhances the contrast and visibility of different tissues, cells, or molecules by applying specific dyes or chemicals to the tissue sections. Histological staining can be effectively used for the diagnosis and prognosis of various diseases, especially cancer. Typically, the most used histological stains are hematoxylin and eosin (H&E), which stains the nuclei of cells, the cytoplasm, and the extracellular matrix. H&E staining can provide a general overview of the tissue morphology and structure. H&E staining may often be used with additional specialty stains to effectively reveal the presence or absence of certain cell types, structures, biomolecules, or microorganisms that are relevant for the diagnosis or research of specific diseases.

However, specialty stains may require more time, additional cost, and might result in a complex histological workflow. Moreover, the variability and inconsistency between different sections due to the misalignment of the tissue region may also be a challenge. Multi-stain histopathology can overcome these limitations allowing simultaneous visualizations of multiple stains on the same tissue section. Multi-stain histopathology can reduce the number of tissue sections needed, simplify the staining protocol, and enabling direct comparison and correlation of different stains. A commonly used method for multi-stain histopathology is immunohistochemistry (IHC), which uses antibodies to detect specific proteins and other antigens in the tissue sections. IHC is a powerful tool for identifying and localizing specific biomarkers that are relevant to a disease state or process and carry rich diagnostic information relevant to diseases. Accurately demultiplexing the IHC images and differentiating the stains can have significant clinical importance in multiplex IHC image analysis. Multi-stain histopathology also poses significant challenges for the image acquisition and analysis.

Brightfield scanners using charge-coupled device (CCD) color camera may be the standard device for image acquisition of histological slides by using white light to illuminate the tissue sections and capture the reflected light using a camera. However, due to the limited resolution of brightfield scanners, multiple stains can appear to occupy the same pixel location. In other words, the complexity of visual inspection-based assessment of multiple stain intensities may increase when they are co-localized in a cell. Thus, limiting the ability of assay designers to correctly determine stain intensities and pathologists, who are generally trained to score single stains, to correctly score multi-stained pixels. The quality and consistency of multi-stain histopathology may be affected by various factors, such as tissue preparation, staining protocol, scanner settings, and environmental conditions. Moreover, due to the limitation of the CCD color camera, the acquired RGB image only contains three channels, and the demultiplexing or unmixing into more than three colors may itself be a challenging task.

### SUMMARY

In some embodiments, a system and method are provided to perform color demultiplexing of brightfield multiplex immunohistochemistry (IHC) images based on a non-linear autoencoder framework. The method includes receiving a multiplex digital pathology image collected using brightfield imaging. The multiplex digital pathology image depicts a slide with a slice of a sample that was stained using multiple IHC markers. The received digital multiplex image is processed by a machine-learning model that is trained on a dataset comprising different multiplex digital pathology images that are stained using different numbers of IHC markers resulting in e.g., singleplex, duplex, triplex images. The machine-learning model may include an encoder (demultiplexing) network that is trained with a decoder (multiplexing) network configured to transform the set of synthetic singleplex images into a synthetic multiplex image. The model is trained using a loss function that is configured such that a calculated loss minimizes a difference between input multiplex digital pathology images and synthetic multiplex images. The trained machine-learning model generates a set of synthetic singleplex images, where each synthetic singleplex image corresponds to an IHC marker. After processing the received multiplex digital pathology image, the trained model may output at least one of the set of synthetic singleplex images.

For dealing with the diverse multiplexing dataset, the method may further include configuration of the encoder (demultiplexing) network to generate the set of synthetic singleplex images conditioned on the number of IHC markers from the multiplex digital pathology image using an attention mechanism. The encoder network may be trained using a loss function that is configured such that a calculated loss scales with a degree of difference between the set of synthetic singleplex images and a set of reference singleplex images, wherein the set of reference singleplex images are adjacent tissue slides stained using single IHC marker of the one or more IHC markers.

It will be appreciated that training may be performed using a loss function that includes one or more loss components. For example, a loss may depend on an extent to which a real multiplex image differs from a synthetic multiplex image and/or an extent to which each of one or more real singleplex images differs from a corresponding synthetic singleplex image.

In some embodiments, the real and/or synthetic multiplex image includes signals corresponding to multiple distinct stains. The distinct stains may target (for example): the Progesterone Receptor (PR), Cal27, or the Estrogen Receptor (ER). Additionally, the real and/or synthetic multiplex images may include a signal from a counterstain (CS) biomarker that is configured to stain nuclei and/or hematoxylin. Hematoxylin can be used as a counterstain to stain the cell nucleus (blue) providing contrast to the specific ER and PR biomarkers. In this case, ER and PR are primarily expressed in the nucleus along with the CS Haematoxylin whereas Cal27 is expressed in the membrane.

In another embodiment, the example of a triplex of cMET_PDL1_EGFR with counterstain comprise of four distinct colors. Here, cMET is stained with carboxytetramethylrhodamine (TAMRA) (pink), programmed death-ligand 1 (PDL1) is stained with benzensulfonyl (Dabsyl) (yellow), epidermal growth factor receptor (EGFR) is stained with Green (green), and the counterstain biomarker is labeled in blue, which is a nuclear stain with hematoxylin. Hematoxylin is used as a counterstain to stain the cell nucleus (blue) providing contrast to the specific biomarkers. PDL1 is expressed in both the membranes and the nucleus, while EGFR localizes to the membrane.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure is described in conjunction with the appended figures:

FIG. 1 is a block diagram illustrating an example overview of a system performing non-linear color demultiplexing of multiplex immunohistochemistry (IHC) based digital pathology images in accordance with an embodiment of the present disclosure.

FIG. 2 illustrates an exemplary network for generating a multiplex digital histopathology image.

FIG. 3 shows an example architecture for color demultiplexing of a multiplex digital pathology image in accordance with some embodiment of the present disclosure.

FIG. 4 shows an illustrative example of a training framework for robust color demultiplexing of a multiplex digital pathology image.

FIG. 5 shows an illustrative example of an objective function from the FIG. 4.

FIG. 6 shows an example flow chart of a method performing color demultiplexing of IHC based multiplex digital pathology image.

FIG. 7A illustrates the results of demultiplexing performance for the input triplex images of two adjacent tissue sections stained using IHC markers, cMET_PDL1_EGFR in accordance with an example implementation of the present disclosure.

FIG. 7B illustrates the results of demultiplexing performance for the input triplex images of other two adjacent tissue sections stained using IHC markers, cMET_PDL1_EGFR in accordance with an example implementation of the present disclosure.

FIG. 8A illustrates the results of demultiplexing performance for the triplex images of three adjacent tissue sections stained using IHC markers, PR_Cal27_ER in accordance with an example implementation of the present disclosure.

FIG. 8B illustrates the results of demultiplexing performance for the triplex images of two other adjacent tissue sections stained using IHC markers, PR_Cal27_ER in accordance with an example implementation of the present disclosure.

### DETAILED DESCRIPTION

In the present disclosure, the term ‘biomarker’ may be defined as a characteristic of tissue such as the presence of a particular cell type, protein, or molecule, especially when indicative of a medical condition.

The term ‘marker’ may be understood in the sense of a stain, dye, or a tag that facilitates the differentiation of a biomarker from surrounding tissue, other biomarkers, or a healthy control sample. The tag, which may be an antibody—specifically one with an affinity for a protein associated with a particular biomarker—can be used for staining or labeling with a fluorophore. A marker may exhibit an affinity for a particular biomarker, e.g., to a particular molecule/protein/cell structure/cell (indicative of a particular biomarker).

Differential staining, fundamental to pathology, encompasses the staining of cytoplasm, cell nuclei, other cell organelles, and specific proteins. A prime illustration may be hematoxylin-eosin (H&E) staining, where hematoxylin (blue) predominantly stains cell nuclei, while eosin (magenta-red) serves as a cytoplasmic stain. The ratio of hematoxylin and eosin staining in the cytoplasm may also provide insights into its basophilia or acidophilia. Another common application of differential staining involves immunohistochemistry (IHC), where multiple unique protein targets may be revealed with different color fluorophores.

For cancer diagnostics, pathologists often rely on brightfield microscopy to evaluate suspicious tissue. This involves employing IHC methods to identify immune cells, evaluate the expression of tumor and cell proliferation markers, and detect conditions like degenerative disorders and infectious diseases. IHC is typically used in clinical oncology to diagnose solid tumors and cytology specimens, and it may incorporate specific staining methods including classic techniques such as H&E staining. Additionally, a diverse array of IHC markers can be utilized to enable accurate diagnosis of various cancer types.

In multiplex immunohistochemistry (IHC), a digital pathology image may be termed e.g., singleplex, duplex, triplex etc. depending on the number of different markers or stains used for staining. For example, singleplex staining may use a single marker or stain on the tissue section for visualization of a specific target or protein. Similarly, in duplex staining two different markers may be used and in triplex staining three different markers may be applied for simultaneously detecting the respective number of different antigens (target proteins) within the single tissue sample. This technique can be used to study multiple biomarkers or antigens in the same tissue section providing comprehensive information about cellular interactions, cell state, heterogeneity, locations, functions, and visualization of these antigens. Such multiplex straining may involve multiple primary antibodies, each recognizing a specific target, and then applying corresponding secondary antibodies labeled with distinct chromogens or fluorophores for visualization. In addition, multiplex staining (e.g., triple staining) saves time compared to three independent staining procedures and preserves valuable samples using less biological material and reagents. With multiplex staining, detection can be done on the same tissue section.

Estrogen is a hormone that can be a contributing factor in breast and endometrial cancer. Estrogen binds to an estrogen receptor (ER) triggering a series of cellular responses that involve proliferation and differentiation of specific cells. Estrogen receptors (ER) and Progesterone receptors (PR) are popular biomarkers used in cancer pathology. ER and PR are nuclear receptors primarily located within the nucleus of a cell, and particularly cancer cells. The staining patterns of ER and PR may help identify the subcellular localization of these biomarkers. For ER, a commonly used antibody is ER-α. The stain is usually visualized with a chromogen e.g., DAB. Progesterone receptor staining may involve the use of antibodies, and the resulting stain may also be visualized with DAB.

Color demultiplexing/unmixing for multiplexed stain images can be both linear and non-linear, depending on the characteristics of staining, specificity of stains, and the method used for unmixing. The linear unmixing methods assume that the spectral response of each stain is independent and additive. The linear algorithms, such as least squares, can be used to separate the contributions of individual stains based on known spectral profiles of the stains. The non-linear unmixing methods are employed when the staining process involves complex interactions and dependencies between different chromogens or fluorophores that are used to label various targets within a tissue such as multiplex IHC images. Such interactions in multiplex staining may arise due to factors such as spectral overlap of different chromogens, complex staining patterns, spatial heterogeneity or non-linear relationship between staining intensity and target concentrations.

Conventional color demultiplexing or unmixing techniques such as color deconvolution or Nonnegative Matrix Factorization (NMF) have limitations and are prone to errors when biomarkers are labeled with fluorophores with overlapping excitation or emission properties. Bleed-through refers to the unwanted overlapping of fluorescent signals from different fluorophores or dyes used to label different cellular or tissue components. Bleed-through can result in inaccurate or misleading data interpretation. Therefore, techniques that accurately and efficiently separate signals from each label are essential. The signal in each channel is conceptualized as a linear combination of contributing staining intensities. Linear unmixing, facilitated by a mixing matrix, can generate an unmixed image. However, a significant drawback can be the requirement to have a reference of pure stains. In the case of a multiplexed brightfield panel with two or three protein biomarkers, overlapping emission spectra may pose challenges. To quantify the signal that is unique to each biomarker, a reference from a tissue section with ‘pure’ biomarker intensities is typically used. However, this approach can be problematic in heterogeneous tissues like breast, colon, lung, brain, etc. and hence may pose a significant challenge when performing advanced multiplexing.

### Overview:

Some embodiments of the present disclosure relate to color demultiplexing of multiplex immunohistochemistry (mIHC) images using an autoencoder-based machine-learning (ML) system trained on a dataset that may include one or more chromogenic dyes resulting in various staining configurations while preserving the biological constraints of the biomarkers. The autoencoder-based ML framework may include: a demultiplexing module (encoder) configured to generate multiple synthetic singleplex images from a given multiplex image, and a multiplexing module (decoder) configured to transform the generated synthetic singleplex images into a synthetic multiplex image. The ML-based system may be trained using a loss function that is configured such that a calculated loss minimizes the difference between input real multiplex digital pathology images and synthetic multiplex images. The trained network may perform a robust separation of multiple staining components into constituent singleplex (Spx) images corresponding to each stain from the multiplex (Mpx) image. The Spx image may refer to an image that represents the visualization of a single staining component or a marker. The term is often used in contrast to a Mpx, which involves the simultaneous visualization of multiple staining components within a single cell or tissue sample.

In some embodiments, the disclosed autoencoder-based system leverages one or more machine-learning models to translate multiplex images to realistic synthetic singleplex images, enabling interpretation by pathologists for delivering diagnosis, conducting semi-quantitative or qualitative assessments of protein expression, and classifying diseases. The ML models are trained on diverse datasets with different staining configurations. Incorporating a combination of triplex (three stains), duplex (two stains) or singleplex (single stains) images into the training data can be beneficial in providing diverse examples for training the model. This increased diversity may lead the framework to capture a broader range of patterns and features and improved generalizability on unseen data, thereby avoiding overfitting and providing an increased task flexibility (e.g., segregation in various staining configurations i.e., triplex, duplex images).

Multiplex IHC images demonstrate the intricacy involved in visually inspecting multiple stain intensities that co-localize within a cell. Demultiplexing of Mpx images becomes more difficult when multiple biomarkers e.g., three or more biomarkers are co-localized. For instance, in breast cancer, the Mpx assay of ER, PR, and human epidermal growth factor receptor 2 (HER2) or ER-PR-HER2 with nuclear staining or hematoxylin counterstain consists of three co-localized biomarkers for nuclear-staining patterns of biomarker ER, PR, and hematoxylin. Moreover, obtaining Spx images corresponding to an Mpx image may be more challenging due to the preparation of formalin-fixed paraffin-embedded (FFPE) slides from a tissue block, which results in multiple slide samples being cut and stained. Consequently, these neighboring tissue slides may inevitably exhibit varying degrees of tissue heterogeneity. In practical terms, samples may be separated by several slides, leading to increased levels of heterogeneity. Hence the reference or ground truth Spx images may have unmatched tissue morphology since the slides are adjacent slides and not the same slide.

To address these problems, a non-linear autoencoder-based methodology is disclosed that leverages one or more machine-learning (ML) models for robust segregation of various staining components of a multiplex (Mpx) image, thereby generating a set of synthetic singleplex (Spx) images. The autoencoder-based system may be trained using an ML-based demultiplexing network (encoder) that is conditioned on the number of IHC markers used for staining of the multiplex images. The encoder network may be trained using a loss function that is configured such that a calculated loss scales with a degree of difference between the set of synthetic singleplex images and a set of reference singleplex images. Therefore, a demultiplexing module may transform the multiplex images into a set of synthetic Spx images that are realistically close to Spx images created on adjacent sections. Subsequently, individual staining elements (or synthetic Spx) are re-multiplexed by deploying another ML model to generate the estimated version of the input Mpx image. The sequence of non-linear color demultiplexing-multiplexing operations is performed in training to achieve a robust generation of synthetic Spx images by following a customized objective function aimed to minimize the structural and morphological differences between the input and the generated output.

To deal with diverse training dataset comprising various staining configurations, the non-linear color demultiplexing network may include a self-attention-based conditional autoencoder (CAE) to generate a corresponding number of synthetic Spx images from the given input Mpx image. The demultiplexing network may further comprise an encoder-decoder architecture trained on the diverse multiplex images to reduce the difference between the output synthetic Spx image and the reference (ground-truth) Spx images created on adjacent sections of tissue. The demultiplexing network may incorporate a conditional input that may correspond to a staining configuration providing context, for example, whether the input is a duplex image or a triplex image. The configuration input may be concatenated along with the multiplex image and fed into the encoder of the demultiplexing module.

The encoder may comprise one or more layers (e.g., convolutional layers, max-pooling, averaging or dense) that gradually down-sample the concatenated input, capturing hierarchical features based on the provided context. The demultiplexing module may utilize self-attention mechanism within one or more encoder layers to focus on relevant spatial regions of the image during encoding process. At each self-attention layer, the model computes attention weights for each pixel based on its relationship with other pixels and/or the contextual information. This allows the model to selectively emphasize important features of the image conditioned on the provided input. The encoder may generate lower-dimensional latent vector by reducing the spatial dimensions of the input, capturing high level features related to the multiplexed content. This encoded information is then passed to the decoder component of the autoencoder.

The conditional input can also be passed directly as encoded information (e.g., as one-hot encoded vector) to the decoder along with the latent representation so the decoder may learn the decoding process conditioned on the provided input. The conditional input may guide the decoder of the demultiplexing module in the reconstruction process. The decoder may comprise convolutional transpose layers that gradually up-sample the latent representation. The skip connections, also called residual connections, facilitate the flow of information between the encoder and decoder. At each up-sampling step, the decoder may concatenate the feature maps from the corresponding layer in the encoder using the skip connections. This concatenation may combine low-level and high-level features, providing the decoder with both local and global details and context. The encoder-decoder network may or may not be symmetric because in the decoder, the model is reconstructing the spatial dimensions by utilizing the skip connections from the encoder that provide contextual information, so the self-attention mechanism may not be as critical. Therefore, the self-attention mechanism may not be incorporated in the decoder layers.

In other embodiments, the non-linear color demultiplexing network may include a self-attention-based conditional variational autoencoder (CVAE) to generate corresponding synthetic Spx images from the given input Mpx image. Unlike regular autoencoder, the encoder maps the input multiplex image to a probabilistic distribution in the latent space, and the decoder reconstructs the input from the samples drawn from this distribution. However, the conditional input may be passed to the encoder and decoder similar to the regular conditional autoencoder.

The output of the demultiplexing module is a set of synthetic singleplex images that corresponds to the conditional input resulting in generation of synthetic Spx images associated with the individual contributions of each color/dye of the Mpx image. For example, if the input is a triplex image, the demultiplexing module is trained to generate three synthetic Spx images, and if the input is a singleplex image the rest of the two output channels will be turned off or omitted.

In some embodiments, the demultiplexing module may be trained in an adversarial manner by combining the conditional autoencoder with a discriminator. When implementing attention mechanisms with autoencoders, it may be important to fine-tune the architecture based on the specific characteristics of the data. The loss function for the demultiplexing module may be a combination of reconstruction loss, such that a calculated loss scales with a degree of difference between the set of synthetic singleplex images, and a set of reference singleplex images and adversarial loss that is introduced by deploying a discriminator alongside the demultiplexing autoencoder. The discriminator, provided with the reference Spx images, acts as a critic of the synthetic Spx images generated from the demultiplexing network (generator). This critic may provide feedback to the generator by distinguishing between real and generated synthetic samples. The feedback of the discriminator introduces a form of regularization to the training process, preventing the generator from overfitting to the training data and enabling it to learn robust and generalizable features. The discriminator takes as an input either a reference Spx image or the synthetic Spx image and distinguishes between real and synthetic images. Simultaneously, the generator (demultiplexing network) is trained to generate synthetic Spx images by minimizing the difference between the synthetic Spx and ground-truth along with the feedback from the discriminator.

For the reconstruction of the multiplex image, a non-linear color multiplexing module may be configured. The multiplexing module may take the synthetic Spx images from the demultiplexing module as input and may deploy a machine-learning model to recover the input multiplexed image. The machine learning model may be a neural network that can generate synthetic multiplex image. In some embodiment, a fully convolutional neural network may be used as an ML model to generate reconstructed multiplex image. In other instances, a combination of convolutional and MLP network may be used as a multiplexing module.

In some embodiment, a U-Net based lightweight autoencoder architecture can be used as a machine-learning model for effectively reconstructing the multiplex image from synthetic singleplex images by leveraging its encoder-decoder architecture with skip connections. At the encoder stage, each synthetic singleplex image may be utilized for capturing features while progressively down-sampling the spatial dimensions. The latent space representation obtained from the encoder may retain crucial information about the input channels. In the decoder, skip connections concatenate feature maps from the corresponding encoder layers, aiding in the reconstruction of spatial details. As the decoder gradually up-samples the latent representation, the U-Net successfully reconstructs the original spatial dimensions, generating a multiplexed image that aggregates the information from the individual singleplex channels. It may also be understood that the attention layers may be introduced in encoder-decoder architecture but are not a necessary requirement.

In an embodiment, the demultiplexing module and multiplexing module may be trained with the respective/individual loss functions and/or by minimizing a combined objective function. The combined (joint) objective function may be a combination of a reconstruction loss between the input multiplex image and the reconstructed multiplex image, and mutual information loss between each pair of the synthetic Spx image. The mutual information may be used in ML tasks such as representation learning to measure the dependence between variables or amount of information shared between variables. Since, the synthetic singleplex images may visually be closer to each other (if not exactly the same); therefore, it may enable learning useful representations particularly in unsupervised learning by measuring the statistical difference between two channels of the Mpx image, capturing meaningful patterns, and relationships in data. Hence, by maximizing mutual information and minimizing the reconstruction loss and adversarial loss, the disclosed model may learn more generalized and robust generation of synthetic Spx images from the diverse dataset of Mpx images (i.e., duplex, triplex, fourplex etc.).

The customized objective may enable learning of the distinct and exclusive features of the cell tissues labeled with multiple stains/dyes enhancing the generalization capabilities and improving proficient demultiplexing performance across a range of situations. The structure of the objective function may also be fine-tuned and optimized to suit diverse real-world experimental conditions, leveraging the training on simulated data. Alternatively, the objective function may be customized to obtain color demultiplexing for the real-world scenarios, where the demultiplexed or Spx ground truth images may not be available. In this setting, after the demultiplexing model is trained on the synthetic data or available small real samples, adversarial loss can be eliminated relying only on reconstruction loss (between the estimated Mpx images and input Mpx images) and the mutual information loss between the estimated Spx images.

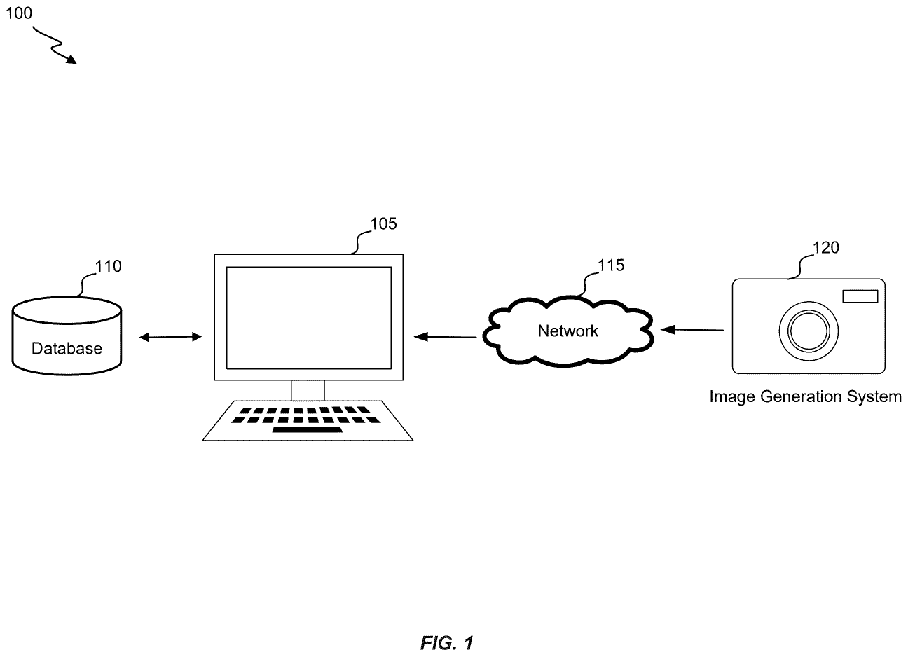

FIG. 1 is a block diagram illustrating an example overview of a system performing non-linear color demultiplexing of multiplex immunohistochemistry (IHC) based histopathology images in accordance with an embodiment of the present disclosure. The exemplary system 100 may include one or more computer systems 105 connected with an image generation system 120 through a network 115. The system 100 may further include one or more databases 110 for the processing and storing of data (e.g., histopathology images). Database 110 may be integral to a memory system on the computer 105 or in secondary storage such as a hard disk, floppy disk, optical disk, or other non-volatile mass storage devices. The computer 105 and the databases 110 may be further connected to one or more communications networks 115. The computer 105 may include a client terminal in communication with one or more servers, or personal digital/data assistants (PDA), laptop computers, mobile computers, internet appliances, one or two-way pagers, mobile phones, or other similar desktop, mobile or hand-held electronic devices.

The computer system 105 of the exemplary system 100 includes a processing system with one or more Central Processing Unit(s) (“CPU”), processors and one or more memories. The computer system 105 may also include a memory for storing a plurality of processing modules or logical instructions that are executed by the one or more processors coupled. The computer memory that stores data may also be maintained on a computer readable medium including magnetic disks, optical disks, organic memory, and any other Volatile (e.g., Random Access Memory (“RAM)) or non-volatile (e.g., Read-Only Memory (“ROM), flash memory, etc.) mass storage system readable by the CPU. The computer readable medium includes cooperating or interconnected computer readable medium, which exist exclusively on the processing system or can be distributed among multiple interconnected processing systems that may be local or remote to the processing system.

The communications network 115 may include, internet, an intranet, a wired Local Area Network (LAN), a wireless LAN (WiLAN), a Wide Area Network (WAN), a Metropolitan Area Network (MAN), Public Switched Telephone Network (PSTN) and other types of communications networks. The communications network 115 may include one or more gateways, routers, or bridges. The communications network 115 may include one or more servers and one or more web-sites accessible by users to send and receive information usable by the one or more computers 105. The one or more servers may also include one or more associated databases for storing electronic information. The communications network 115 includes, but is not limited to, data networks using the Transmission Control Protocol (TCP), User Datagram Protocol (UDP), Internet Protocol (IP) and other data protocols.

Besides processors and memory, the computer system 105 may also include user input and output devices such as a keyboard, mouse, stylus, and a display/touchscreen. For instance, the computer system 105 may provide a means for inputting image data from one or more scanned IHC slides to memory. Image data may include data related to color channels or frequency channels. A biological specimen, for example a tissue section, may need to be stained for biomarkers associated with chromogenic stains for brightfield imaging or fluorophores for fluorescence imaging. Staining assays can use chromogenic stains for brightfield imaging, or combinations of organic fluorophores, synthetic fluorophores, or quantum dots for fluorescence imaging. In the analysis of biological specimens, different stains may be specified to identify one or more types of biomarkers.

The term ‘sample’ may be understood as material derived from a biological organism, comprising but not limited to hair, skin samples, tissue samples, cultured cells, cultured cell media, and biological fluids. The term ‘tissue’ refers to a mass of interconnected cells (e.g., lung tissue, neural tissue, or eye tissue) derived from a human or other animal and includes the connecting material and the liquid material in association with the cells. In the context of histopathology, the term “slide” refers to a glass microscope slide carrying a thin section of tissue that has been stained for microscopic examination. The term ‘sample’ also includes media containing isolated cells. One skilled in the art may determine the quantity of samples required to obtain a reaction by standard laboratory techniques.

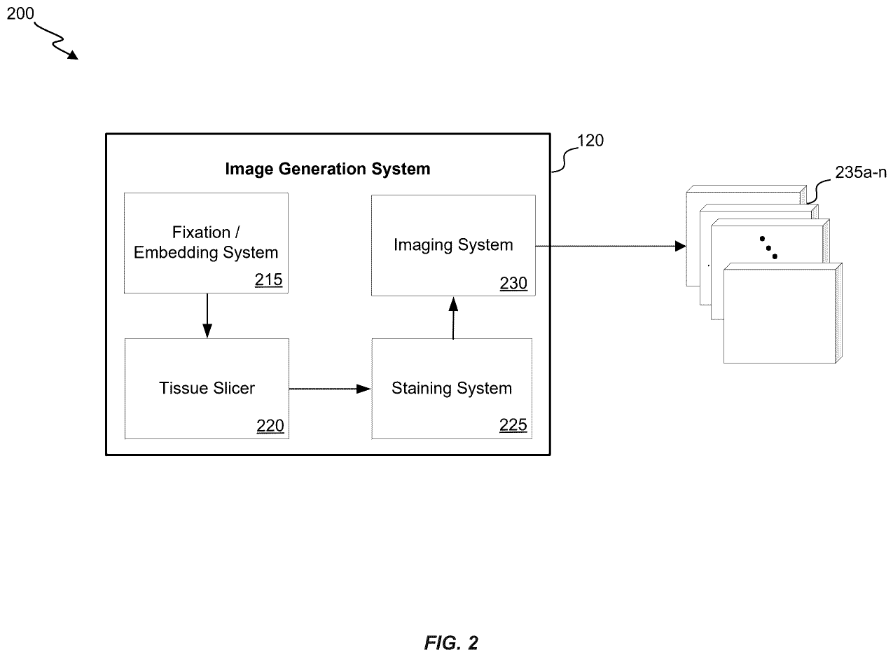

FIG. 2 shows an exemplary network of a digital pathology image generation system from FIG. 1. Images are generated by an image generation system 120. A fixation/embedding system 215 fixes and/or embeds a tissue sample (e.g., a liquid fixing agent, such as formaldehyde solution) and/or an embedding substance (e.g., a historical wax, such as paraffin wax and/or one or more resins, such as styrene or polyethylene). Each slice may be fixed by exposing the slice to a fixating agent for a predefined period of time (e.g., at least 3 hours) and by then dehydrating the slice (e.g., via exposure to an ethanol solution and/or a clearing intermediate agent). The embedding substance can infiltrate the slice when it is in liquid state (e.g., when heated).

A tissue slicer 220 then slices the fixed and/or embedded tissue sample (e.g., a sample of a tumor) to obtain a series of sections, with each section having a thickness of, for example, 4-5 microns. Such sectioning can be performed by first chilling the sample and then slicing the sample in a warm water bath. The tissue can be sliced using (for example) a vibratome or compresstome.

Because the tissue sections and the cells within them are virtually transparent, preparation of the slides typically includes staining (e.g., automatically staining) the tissue sections to render relevant structures more visible. In some instances, the staining is performed manually. In some instances, the staining is performed semi-automatically or automatically using a staining system 225.

The staining can include exposing an individual section of the tissue to one or more different stains (e.g., consecutively, or concurrently) to reveal different characteristics of the tissue. For example, each section may be exposed to a predefined volume of a staining agent for a predefined period of time. The staining agent can include (for example) an RNA probe, protein probe (e.g., nuclear-protein probe or cytoplasm-protein probe), an immunohistochemistry stain, a probe for a secreted substance, etc. In some instances, the staining agent is one that stains for KAPPA mRNA or LAMBDA mRNA.

One exemplary type of tissue staining is histochemical staining, which uses one or more chemical dyes (e.g., acidic dyes, basic dyes) to stain tissue structures. Histochemical staining may be used to indicate general aspects of tissue morphology and/or cell microanatomy (e.g., to distinguish cell nuclei from cytoplasm, to indicate lipid droplets, etc.). One example of a histochemical stain is hematoxylin and eosin (H&E). Other examples of histochemical stains include trichrome stains (e.g., Masson's Trichrome), Periodic Acid-Schiff (PAS), silver stains, and iron stains. The molecular weight of a histochemical staining reagent (e.g., dye) is typically about 500 kilodaltons (kD) or less, although some histochemical staining reagents (e.g., Alcian Blue, phosphomolybdic acid (PMA)) may have molecular weights of up to two or three thousand kD. One case of a high-molecular-weight histochemical staining reagent is alpha-amylase (about 55 kD), which may be used to indicate glycogen.

Another type of tissue staining is immunohistochemistry (IHC, also called “immunostaining”), which uses a primary antibody that binds specifically to a target antigen of interest (biomarker). IHC may be direct or indirect. In direct IHC, the primary antibody is directly conjugated to a label (e.g., a chromophore or fluorophore). In indirect IHC, the primary antibody is first bound to the target antigen, and then a secondary antibody that is conjugated to a label (e.g., a chromophore or fluorophore) is bound to the primary antibody. The molecular weights of IHC reagents are much higher than those of histochemical staining reagents, as the antibodies have molecular weights of about 150 kD or more.

The sections may then be individually mounted on corresponding slides, which an imaging system 230 can then scan to generate raw multiplex digital-pathology images 235a-n. Each section may be mounted on a slide, which is then scanned to create a digital image that may be subsequently examined by digital pathology image analysis and/or interpreted by a human pathologist (e.g., using image viewer software). The imaging may include capturing bright-field images of the slide section.

In some instances, a pathologist or other expert may review and manually annotate the digital images of the slides (e.g., tumor area, necrosis, etc.). In some instances, annotation of regions of interest are performed automatically using a computer-vision technique. Some of the digital-pathology images 235a-n may be used by a color demultiplexing system 300.

A digital histopathology image (e.g., 235a) typically includes an array, usually a rectangular matrix, of pixels. Each ‘pixel’ is one picture element and is a digital quantity that is a value that represents some property of the image at a location in the array corresponding to a particular location in the image. Typically, in monochrome tone black and white images the pixel values represent a gray scale value. Pixel values for a digital image typically conform to a specified range. For example, each array element may be one byte (i.e., eight bits) representing pixel values in the range of 0 to 255. In a gray scale image, a 255 may represent absolute white and zero total black (or visa-versa). Color images consist of three-color planes, generally corresponding to red, green, and blue (RGB). For a particular pixel, there is one value for each of these color planes, (i.e., a value representing the red component, a value representing the green component, and a value representing the blue component). By varying the intensity of these three components, all colors in the color spectrum typically may be created.

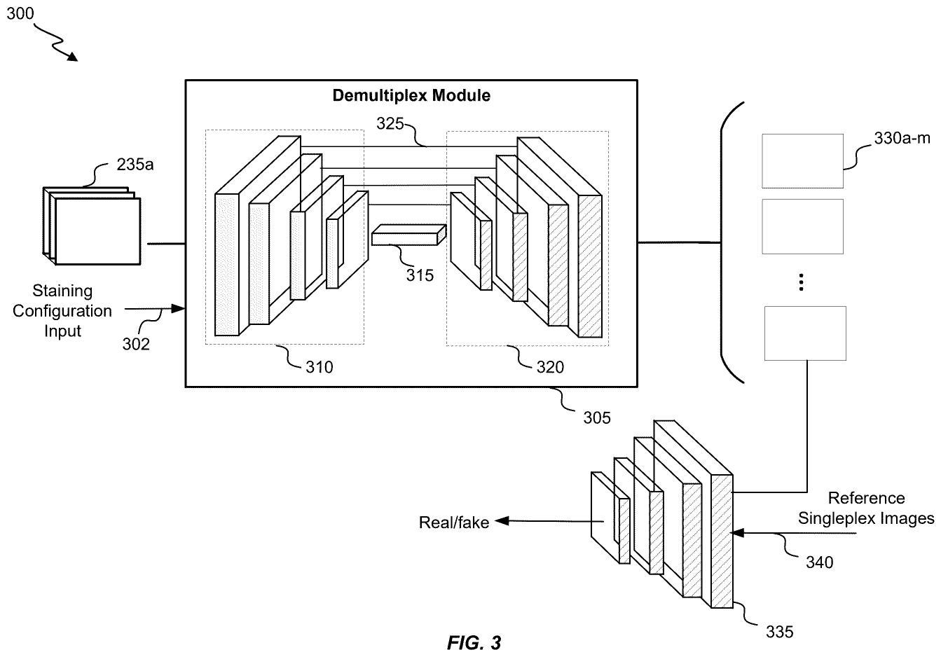

FIG. 3 shows an example architecture of a non-linear conditional autoencoder-based color demultiplexing framework 305 in accordance with some embodiment of the present disclosure. The demultiplexing module 305 takes Mpx image (y) 235a, generated from the image generation system 120 along with the staining configuration input (c) 302 that provides information/context whether the input multiplex image is a triplex, duplex or singleplex. This configuration input 302 may be provided to the encoder as an encoded vector, for example, as one hot encoded vector concatenated with the input multiplex image. The demultiplexing module 305 may include the conditional autoencoder architecture integrated with a self-attention mechanism. The conditional autoencoder 315 comprises an encoder 310 component that reduces the dimensionality of the input Mpx image (e.g., 235a).

The demultiplexing encoder 310 may comprise one or more layers e.g., convolutional layers, max-pooling etc. that gradually down-sample the input, capturing hierarchical features conditioned on input. The demultiplexing module 305 may utilize a self-attention mechanism within one or more encoder layers to focus on relevant spatial regions of the image during encoding process. At each self-attention layer, the model computes attention weights for each pixel based on its relationship with other pixels and the context information from the conditional input. This allows the model to selectively emphasize important features based on the type of multiplex image. The encoder 310 reduces the spatial dimensions of the input, capturing abstract features related to the multiplexed content.

The output of the demultiplexing encoder DE (h|y, c) 310 is a lower-dimensional latent space representation (h) 315 that encodes spatial and spectral information from the multiplexed input image 235 along with the configurational input 302. This combined encoded information is then passed to the demultiplexing decoder component DD(ŷ|h, c) 320 of the autoencoder. The U-net decoder 320 may comprise convolutional transpose layers that gradually up-sample the latent representation 315. The configurational input 302 may guide the decoder in the reconstruction process for reconstructing synthetic one or more singleplex images. The skip connections 325, also called residual connections facilitate the flow of information between the encoder 310 and decoder 320.

At each up-sampling step, the demultiplexing decoder 320 concatenates the feature maps from the corresponding layer in the encoder 310 using the skip connections 325. This concatenation may combine low-level and high-level features, providing the decoder with both local and global details and context. In the decoder 320, where the model is reconstructing the spatial dimensions, the skip connections 325 from the encoder provide contextual information, so the self-attention mechanism may not be as critical. Therefore, the self-attention mechanism may or may not be incorporated in the decoder layers 320. The output of the demultiplexing module 305 is a set of synthetic singleplex images 330a-m corresponding to the individual contributions of each color/dye of the Mpx image conditioned on the staining configuration input.

Crafting a loss function for a demultiplexing module with a self-attention-based conditional autoencoder 305 may involve a combination of terms to encourage accurate reconstruction, for example, α_{recon}L_{recon}+α_{cond}L_{cond}. The reconstruction loss term (L_{recon}) may enable the autoencoder to accurately reconstruct the singleplex (Spx) images from the channel combinations by minimizing the difference between the output synthetic Spx images and reference Spx images. The reference Spx images may have unmatched tissue morphology due to fact that the slides are adjacent slides, not the same slide. Thus, tissue morphology differences may be present. A common choice may be mean square error (MSE):

$$\frac{1}{N} {\sum }_{n = 1}^{N} {‖ X - \hat{X} ‖}^{2}$$

or mean absolute error (MAE), where X is the reference (or ground truth) Spx image, \hat{X} is the reconstructed or estimated output Spx image and N is the number of samples in the training dataset. Using MSE as a loss function can emphasize accurate pixel-wise similarity for generating Spx images to closely match the original Mpx images in terms of pixel intensity.

In addition to MSE, an additional term, structural similarity index (SSI) may be added to minimize the difference for image-related tasks. SSI is a metric that quantifies the similarity between two images, considering luminance, contrast, and structure. It provides a more perceptually meaningful measure than pixel-wise differences. For the demultiplexing, SSI loss may be defined as: 1-SSI(X, \hat{X}). SSI metric is normalized between 0 and 1, where 1 represents that two images are similar. Alternatively, perceptual loss can also be combined that leverages the power of pre-trained convolutional neural networks (CNNs) to compare high-level features between the target and the output images, rather than pixel-level differences. Hence, for crafting the loss function for the demultiplexing module, MSE, SSI, perceptual loss or a combination thereof can be used along with the respective regularization terms (e.g., α_{recon}) to generate synthetic Spx images from Mpx images.

The conditional loss can be based on the condition, for example, MSE of input configuration input and the estimated configuration at the output of demultiplexing module, i.e.,

$$\frac{1}{N} {\sum }_{n = 1}^{N} {‖ c - \hat{c} ‖}^{2} .$$

It may quantify the error associated with the conditional criterion encouraging the model to leverage the provided context effectively. It may also be termed as supervised loss. The weight regularization terms such as α_{recon} and α_{cond} may be added to the objective function to prevent overfitting and to control the complexity of the model.

In one instance, the demultiplexing module 305 may be trained in an adversarial manner by combining the conditional autoencoder with a discriminator 335 as shown in FIG. 3. When incorporating attention mechanisms into autoencoders, it may be beneficial to tailor the architecture to the specific characteristics of the data. The loss function for the demultiplexing module 305 can be a composite of reconstruction loss. This loss scales with the disparity between sets of synthetic singleplex images 330a-m and a reference set of singleplex images 340. Additionally, an adversarial loss is introduced by deploying a discriminator 335 alongside the demultiplexing autoencoder 305.

The discriminator 335, provided with reference Spx images, acts as a critic for the synthetic Spx images generated by the demultiplexing network 305. This critic may provide feedback to the demultiplexing network 305 by discerning between real and generated synthetic samples. This feedback from the discriminator 335 serves as a form of regularization during the training process, preventing the generator 305 from overfitting to the training data and promoting the learning of robust and generalizable features. The discriminator 335 assesses either a reference Spx image 340 or a synthetic Spx image 330, distinguishing between real and synthetic images. Concurrently, the demultiplexing network is trained to produce synthetic Spx images by minimizing the difference between the synthetic Spx 330 and ground truth 340, incorporating the feedback received from the discriminator 335. The loss function for the adversarial training may be formulated as:

$$\frac{1}{N} {{\sum }_{i = 1}^{N} [ 1 - D ( {D}_{DM} ( {y}_{i} \vert c ) ) ]}^{2} ,$$

where D_{DM} is the demultiplexing module.

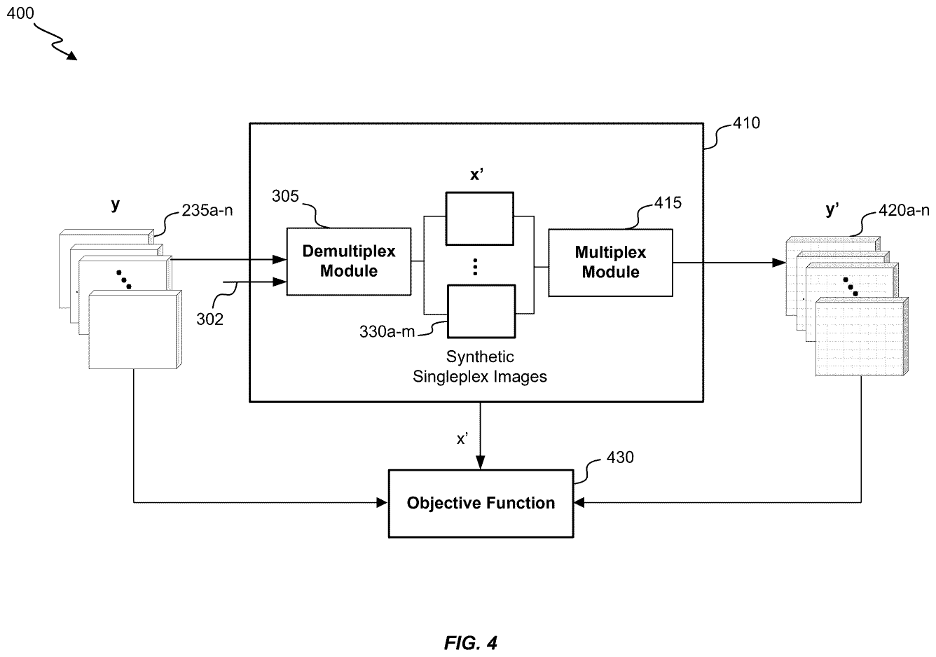

FIG. 4 shows an illustrative example of a training framework 400 for robust demultiplexing of multiplexed digital pathology image in accordance with an embodiment of the present disclosure. The robust learning framework 305 may include a demultiplexing/unmixing module 305 and multiplexing module 415 connected sequentially. The framework may obtain color demultiplexing for the real-world setting where the corresponding demultiplexed or Spx reference images 425 may not be available. The demultiplexing module 305 is configured to generate individual synthetic Spx images 330a-m from an Mpx image (e.g., 235a) where each synthetic Spx image may correspond to a single stain. The output from the demultiplexing module 305 is fed into the multiplexing module 415 that takes these synthetic Spx images 330a-m and reconstructs an estimate of the input Mpx image 235a-n. The non-linear demultiplexing 305 and multiplexing modules 415 may iteratively improve the color unmixing of Mpx images 235a-n by satisfying the objective function 430.

The multiplexing module 415 may take the synthetic Spx images 320a-m from the demultiplexing module 305 as input and may deploy a machine-learning model to recover the input multiplexed image. The machine-learning model may include a lightweight U-Net architecture for effectively reconstructing the multiplex image from synthetic singleplex images 320a-m by leveraging its encoder-decoder structure with skip connections. At encoder stage, each synthetic singleplex image 320 may be treated as an individual channel for capturing features from these channels while progressively down-sampling the spatial dimensions. The latent space representation obtained from the encoder may retain crucial information about the input channels. In the decoder, skip connections concatenate feature maps from the corresponding encoder layers, aiding in the reconstruction of spatial details. As the decoder gradually up-samples the latent representation, the U-Net successfully reconstructs the original spatial dimensions, generating a multiplexed image that amalgamates information from the individual singleplex channels.

Similar to demultiplexing module, the training objective may involve minimizing a reconstruction loss, such as mean absolute error (MAE) or mean squared error (MSE) along with structural similarity index (SSI) or perceptual loss, enabling the generated multiplex image 420a-n to be closely matched with the target multiplex image 235a-n. Hence, for the multiplexing module, the MSE loss term can be written as

$$\frac{1}{N} {\sum }_{n = 1}^{N} {‖ Y - \hat{Y} ‖}^{2}$$

and SSI loss term can be reformulated as, 1-SSI(Y, Ŷ). where Y is the reference or target multiplex image, Ŷ is the reconstructed or estimated Mpx image and N is the number of samples in the training dataset. Fine-tuning hyperparameters may enhance the ability of the U-Net to accurately reconstruct high-quality multiplex images 420a-n from the given synthetic singleplex 320a-m inputs.

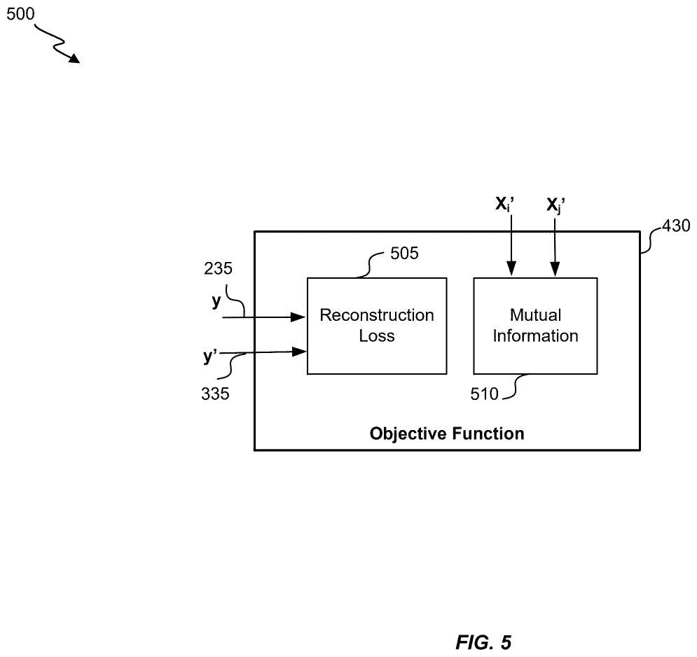

FIG. 5 illustrates an example representation of the joint objective function 430 for the color demultiplexing of multiplex IHC images in accordance with an embodiment of the present disclosure. It should be understood that the demultiplexing module and the multiplexing module may be trained with the individual loss functions as discussed above and/or can be jointly trained with the objective function. The objective function 430 may be combined with the reconstruction loss 605 between the input Mpx image 235 and estimated Mpx image 335 (from the multiplexing network 415) and mutual information loss between each pair of the synthetic Spx image. The mutual information may enable learning useful representations particularly in unsupervised learning by measuring the statistical difference between two channels of the Mpx image, capturing meaningful patterns and relationships in data. Hence, by maximizing mutual information and minimizing the reconstruction loss, the disclosed model may learn more generalized and robust generation of synthetic Spx images from the diverse dataset of Mpx images (i.e., duplex, triplex, fourplex etc.). The customized objective may enable learning of the distinct and exclusive features of the cell tissues labeled with multiple stains/dyes enhancing the generalization capabilities and improving proficient demultiplexing performance across a range of situations.

The structure of the objective function may also be fine-tuned and optimized to suit diverse real-world experimental conditions, leveraging the training on simulated data. Alternatively, the objective function may be customized to obtain color demultiplexing for the real-world scenarios where the demultiplexed or Spx ground truth images may not be available. In this setting, after the demultiplexing model is trained on the synthetic data, adversarial loss can be eliminated from the demultiplexing module relying only on reconstruction loss (between the estimated Mpx images and input Mpx images) and the mutual information loss between the estimated Spx images.

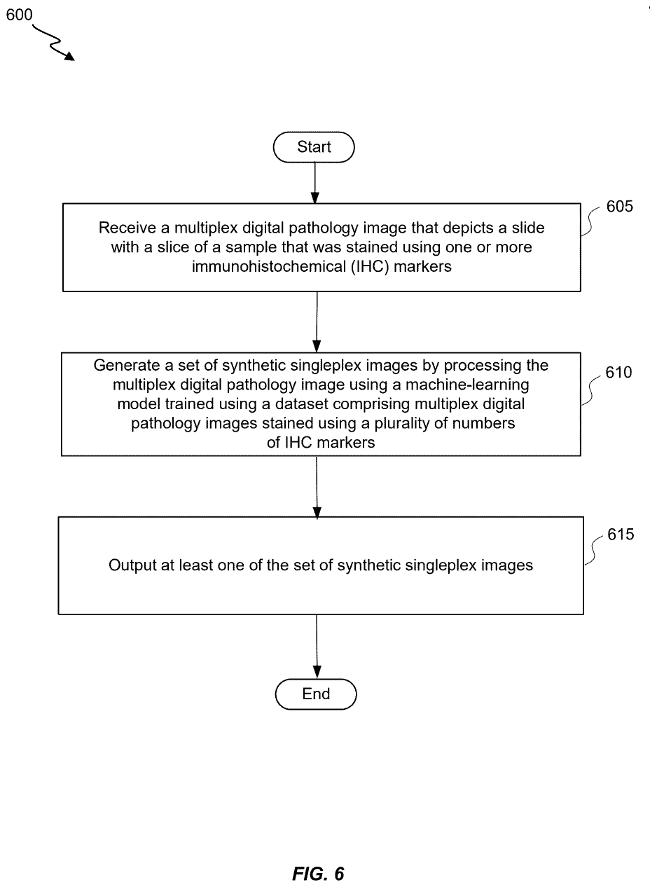

FIG. 6 shows an example flow chart of a computer-implemented method performing color demultiplexing of IHC based multiplex digital pathology image. At block 605, a multiplex digital pathology image is received that depicts a slide with a slice of a sample that was stained using one or more immunohistochemical (IHC) markers. For example, using multiple IHC markers the resulting multiplex digital pathology image may be a singleplex, duplex or a triplex image. At block 610, a machine-learning model may be trained on a diverse dataset comprising various staining configurations resulting in a dataset that may include e.g., duplex, triplex or singleplex images. The trained machine-learning model may process the received multiplex digital pathology image and may generate a set of synthetic singleplex images. Each synthetic singleplex image of the set of synthetic singleplex images corresponds to an IHC marker of the one or more IHC markers. The trained machine-learning model may include an encoder network that was trained with a decoder network configured to transform the set of synthetic singleplex images conditioned on the configurational input into a synthetic multiplex image. The training used a loss function configured such that a calculated loss scales with a degree of difference between input multiplex digital pathology images and synthetic multiplex images. Finally, at block 615, at least one of the set of synthetic singleplex images is output.

### Example Implementation

An example implementation of the framework is provided for color demultiplexing of the multiplex histopathology images using non-linear autoencoder based framework as illustrated in FIG. 4. This approach proves to effectively color demultiplex the multiplex histopathology images into substituent singleplex images.

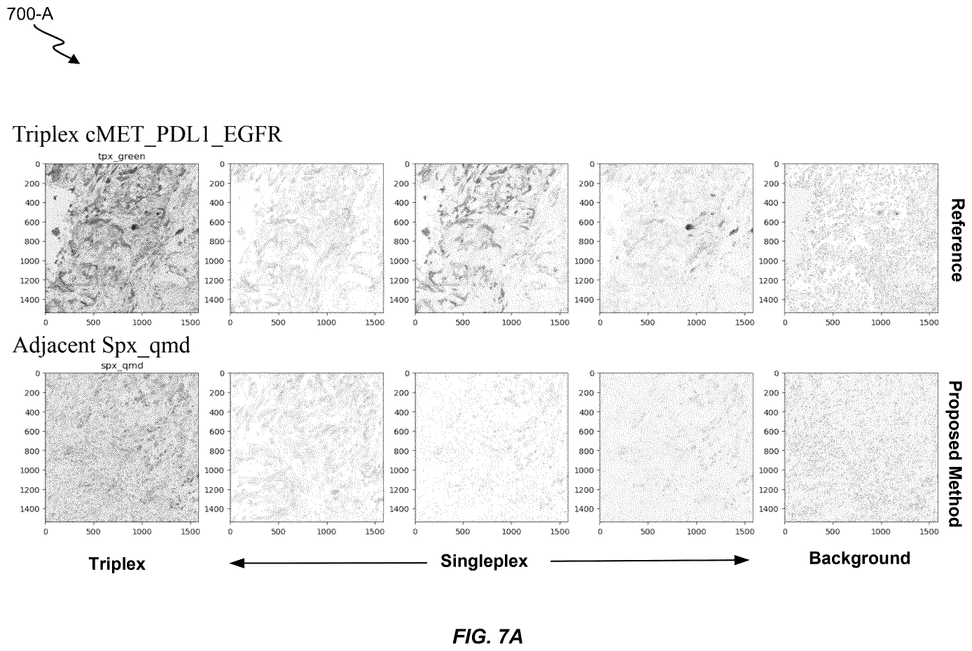

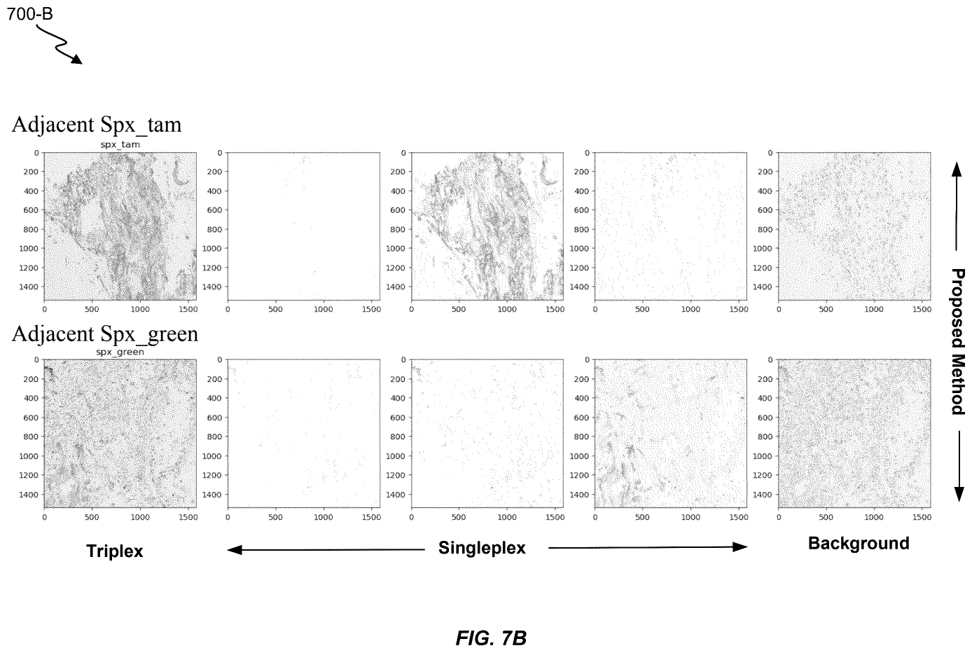

In the following demonstration, IHC is used to stain four adjacent serial tissue sections with four markers per tissue section as illustrated in first column of FIG. 7. Each marker is designated by a specific color. The rest of the columns represent singleplex (Spx) images, each representing a single marker or IHC stain. The first column from the right of FIG. 7 represents background or any non-specific signal in the triplex image. This image is generated during the demultiplexing process to capture elements that do not correspond to the specific stains of interest. The background Spx image may help identify and separate background noise from the actual staining signals. The results of demultiplexing performance for the input triplex images of two adjacent tissue sections stained using IHC markers, cMET_PDL1_EGFR in accordance with an example implementation of the present disclosure is shown in FIG. 7A. The example of a triplex of cMET_PDL1_EGFR with counterstain comprise of four distinct colors. For each color, cMET was stained with carboxytetramethylrhodamine (TAMRA) shown in pink, programmed death-ligand 1 (PDL1) was stained with benzensulfonyl (Dabsyl) shown in yellow, epidermal growth factor receptor (EGFR) was stained with Green shown in green, and counterstain biomarker in blue, which was nuclear staining with hematoxylin. Hematoxylin is used as a counterstain to stain the cell nucleus (blue) providing contrast to the specific biomarkers. PDL1 is expressed in both the membrane and the nucleus, and EGFR is in the membrane.

The reference singleplex (SPx) images (ground-truth) from single-marker IHC in serial tissue sections are shown in the first row of FIG. 7A. The reference images are the corresponding adjacent SPx images referring as the intensity ground truth. The tissue morphology is not matched due to fact that they are adjacent slides, not the same slide. Thus, there remains tissue morphology differences. Comparing the stain intensity levels, it can be observed that the image quality and stain intensities of the generated and real adjacent images look similar for all three SPx images. The second row of FIG. 7A shows performance for the color demultiplexing of adjacent triplex to Spx qmd.

FIG. 7B illustrates the results of demultiplexing performance for the input triplex images of other two adjacent tissue sections stained using IHC markers, cMET_PDL1_EGFR in accordance with an example implementation of the present disclosure. In the first row of FIG. 7B, the performance is shown for the color demultiplexing of triplex to Spx TAMRA. Similarly, the second row shows performance of the disclosed framework for adjacent input triplex to Spx green.

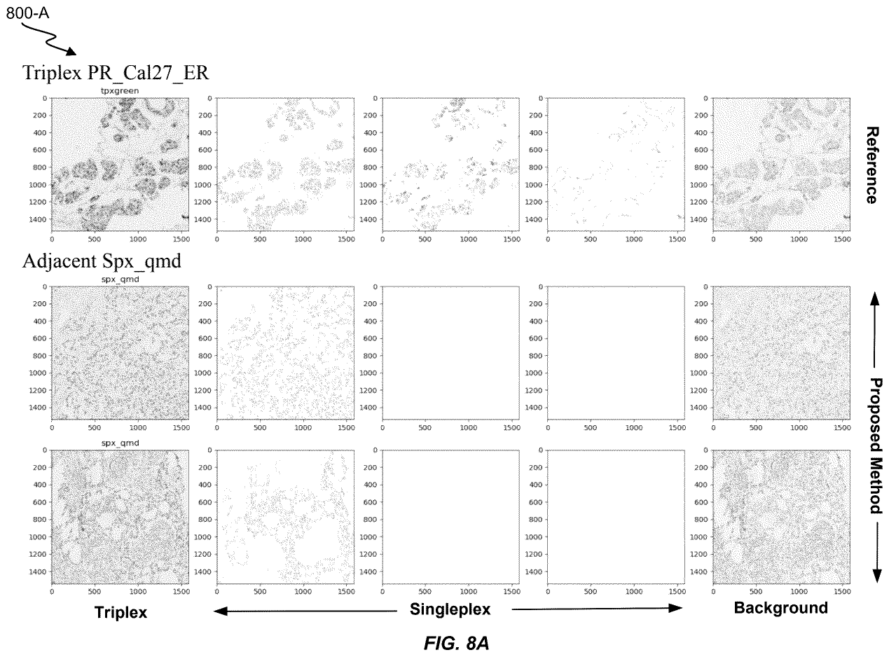

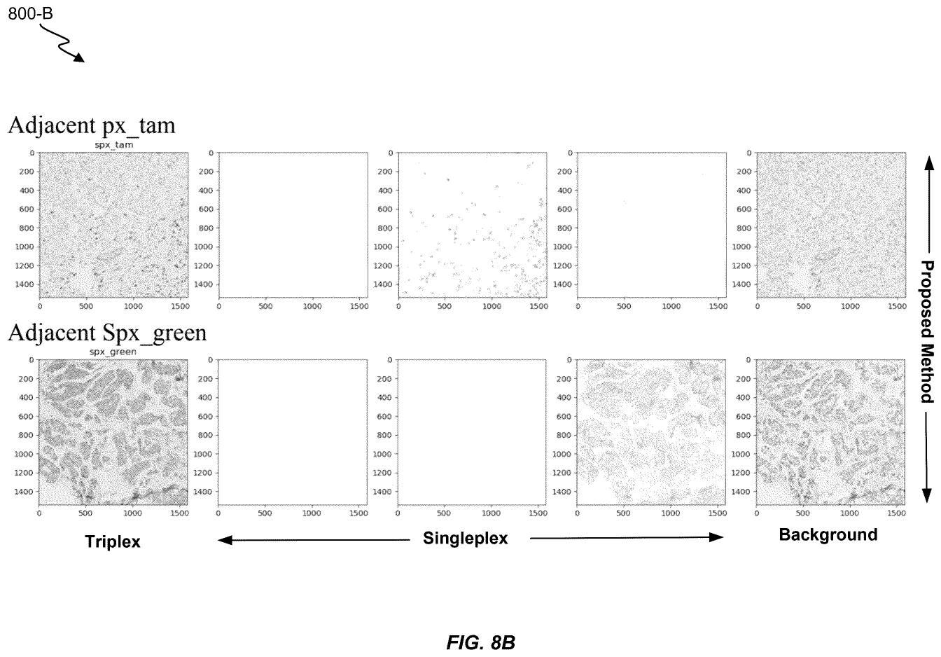

In the following demonstration, IHC is used to stain five adjacent serial tissue sections with four markers per tissue section as illustrated in first column of FIG. 8, where each marker is designated by a specific color. FIG. 8A illustrates the results of demultiplexing performance for the triplex images of three adjacent tissue sections stained using IHC markers, PR_Cal27 ER in accordance with an example implementation of the present disclosure. The example of a triplex of PR_Cal27_ER with counterstain comprise of four distinct colors. For each color, PR was stained with carboxytetramethylrhodamine (TAMRA) shown in pink, Cal27 was stained with benzensulfonyl (Dabsyl) shown in yellow, ER was stained with Green shown in green, and counterstain biomarker in blue, which was nuclear staining with hematoxylin. Hematoxylin is used as a counterstain to stain the cell nucleus (blue) providing contrast to the specific biomarkers. In this case, ER and PR are primarily expressed in the nucleus along with the CS Haematoxylin, and Cal27 is expressed in the membrane. The second and third row of FIG. 8A shows performance for the color demultiplexing of adjacent triplex image to Spx qmd (yellow).

FIG. 8B illustrates the results of demultiplexing performance for the triplex images of other two adjacent tissue sections stained using IHC markers, PR_Cal27_ER in accordance with an example implementation of the present disclosure. The first row of FIG. 8B shows performance for the color demultiplexing of adjacent triplex image to Spx TAMRA (pink). Similarly, the second row of FIG. 8B shows performance for the color demultiplexing of adjacent triplex image to Spx Green (green).

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
receiving a multiplex digital pathology image that depicts a slide with a slice of a sample that was stained using one or more immunohistochemical (IHC) markers, wherein the multiplex digital pathology image was collected using brightfield imaging;
generating a set of synthetic singleplex images by processing the multiplex digital pathology image using a machine-learning model trained using a dataset that includes multiplex digital pathology images stained using a plurality of numbers of IHC markers, wherein each synthetic singleplex image of the set of synthetic singleplex images corresponds to an IHC marker of the one or more IHC markers, and wherein the machine-learning model is an encoder network that was trained with a decoder network configured to transform the set of synthetic singleplex images into a synthetic multiplex image; and
outputting at least one of the set of synthetic singleplex images.

2. The computer-implemented method of claim 1, wherein the machine-learning model is trained using a loss function configured such that a calculated loss minimizes a difference between input multiplex digital pathology images and synthetic multiplex images.

3. The computer-implemented method of claim 1, wherein the encoder network is configured to generate the set of synthetic singleplex images, conditioned on a number of IHC markers, from the multiplex digital pathology image using an attention mechanism.

4. The computer-implemented method of claim 3, wherein the encoder network is trained using a loss function that is configured such that a calculated loss scales with a degree of difference between the set of synthetic singleplex images and a set of reference singleplex images, wherein the set of reference singleplex images are adjacent tissue slides stained using single IHC marker of the one or more IHC markers.

5. The computer-implemented method of claim 1, wherein:
the one or more IHC markers include: a Hematoxylin counterstain, a marker for Cal27, and markers to label estrogen receptors (ER) and progesterone receptors (PR).

6. The computer-implement method of claim 1, wherein:
the one or more IHC markers include: a Hematoxylin and Eosin (H&E) counterstain, a marker for the receptor tyrosine kinase c-MET, a marker for the protein programmed death-ligand 1 (PDL1), and a marker for the epidermal growth factor receptor (EGFR).

7. A system comprising:
one or more data processors; and
a non-transitory computer readable storage medium containing instructions which, when executed on the one or more data processors, cause the one or more data processors to perform a set of actions including:
receiving a multiplex digital pathology image that depicts a slide with a slice of a sample that was stained using one or more immunohistochemical (IHC) markers, wherein the multiplex digital pathology image was collected using brightfield imaging;
generating a set of synthetic singleplex images by processing the multiplex digital pathology image using a machine-learning model trained using a dataset that includes multiplex digital pathology images stained using a plurality of numbers of IHC markers, wherein each synthetic singleplex image of the set of synthetic singleplex images corresponds to an IHC marker of the one or more IHC markers, and wherein the machine-learning model is an encoder network that was trained with a decoder network configured to transform the set of synthetic singleplex images into a synthetic multiplex image; and
outputting at least one of the set of synthetic singleplex images.

8. The system of claim 7, wherein the machine-learning model is trained using a loss function configured such that a calculated loss minimizes a difference between input multiplex digital pathology images and synthetic multiplex images.

9. The system of claim 7, wherein the encoder network is configured to generate the set of synthetic singleplex images, conditioned on a number of IHC markers, from the multiplex digital pathology image using an attention mechanism.

10. The system of claim 9, wherein the encoder network is trained using a loss function that is configured such that a calculated loss scales with a degree of difference between the set of synthetic singleplex images and a set of reference singleplex images, wherein the set of reference singleplex images are adjacent tissue slides stained using single IHC marker of the one or more IHC markers.

11. The system of claim 7, wherein:
the one or more IHC markers include: a Hematoxylin counterstain, a marker for Cal27, and markers to label estrogen receptors (ER) and progesterone receptors (PR).

12. The system of claim 7, wherein:
the one or more IHC markers include: a Hematoxylin and Eosin (H&E) counterstain, a marker for the receptor tyrosine kinase c-MET, a marker for the protein programmed death-ligand 1 (PDL1), and a marker for the epidermal growth factor receptor (EGFR).

13. A computer-program product tangibly embodied in a non-transitory machine-readable storage medium, including instructions configured to cause one or more data processors to perform a set of actions including:
receiving a multiplex digital pathology image that depicts a slide with a slice of a sample that was stained using one or more immunohistochemical (IHC) markers, wherein the multiplex digital pathology image was collected using brightfield imaging;
generating a set of synthetic singleplex images by processing the multiplex digital pathology image using a machine-learning model trained using a dataset that includes multiplex digital pathology images stained using a plurality of numbers of IHC markers, wherein each synthetic singleplex image of the set of synthetic singleplex images corresponds to an IHC marker of the one or more IHC markers, and wherein the machine-learning model is an encoder network that was trained with a decoder network configured to transform the set of synthetic singleplex images into a synthetic multiplex image; and
outputting at least one of the set of synthetic singleplex images.

14. The computer-program product of claim 13, wherein the machine-learning model is trained using a loss function configured such that a calculated loss minimizes a difference between input multiplex digital pathology images and synthetic multiplex images.

15. The computer-program product of claim 13, wherein the encoder network is configured to generate the set of synthetic singleplex images, conditioned on a number of IHC markers, from the multiplex digital pathology image using an attention mechanism.

16. The computer-program product of claim 15, wherein the encoder network is trained using a loss function that is configured such that a calculated loss scales with a degree of difference between the set of synthetic singleplex images and a set of reference singleplex images, wherein the set of reference singleplex images are adjacent tissue slides stained using single IHC marker of the one or more IHC markers.

17. The computer-program product of claim 13, wherein:
the one or more IHC markers include: a Hematoxylin counterstain, a marker for Cal27, and markers to label estrogen receptors (ER) and progesterone receptors (PR).

18. The computer-program product of claim 13, wherein:
the one or more IHC markers include: a Hematoxylin and Eosin (H&E) counterstain, a marker for the receptor tyrosine kinase c-MET, a marker for the protein programmed death-ligand 1 (PDL1), and a marker for the epidermal growth factor receptor (EGFR).
